(19) United States
(12) Patent Application Publication
(10) Pub. No.: US 2008/0193547 A1
(43) Pub. Date: Aug. 14, 2008

(54) Polymeric nanoparticles by ion-ion Interactions

(76) Inventors: Janos Borbely, Debrecen (HU); Magdolna Bodnar, Hajduboszormeny (HU); Istvan Hajdu, Tiszacsege (HU); John F. Hartmann, Princeton Jet, NJ (US)

Correspondence Address: Thomas A. O'Rourke, Bodner & O'Rourke, 425 Broadhollow Road, Melville, NY, 11747, US

(21) Appl. No.: 12/005,643

(22) Filed: Dec. 27, 2007

Related U.S. Application Data
(60) Provisional application No. 60/877,258, filed on Dec. 27, 2006.

(51) Int. Cl. A61K 9/16; A61K 31/7088; A61K 31/519

(52) U.S. Cl. 424/499; 424/489; 514/44; 514/249

(57) ABSTRACT

The present invention relates to biocompatible and biodegradable, stimuli sensitive, polymeric nanoparticles, which are formed by ion-ion interaction in aqueous media. Synthetic and biological macromolecules with ionizable functional groups are capable of forming nanoparticles whose size and surface properties are sensitive to environmental factors such as pH, temperature and salt concentration. Nanodevices made from these nanoparticles are designed for therapeutic applications included but not limited to use as drug carriers and/or used as contrast agents in MRI diagnosis and the like. The adjustable size of the nanodevices and their stimuli sensitivity allows specific delivery applications. Thus, these nanosystems are potential carrier tools for delivery of active ingredients such as drugs, as well as DNA, RNA, siRNA for cosmetics, pharmaceutical applications, etc.

This application claims priority on U.S. Application Ser. No. 60/877,258 filed Dec. 27, 2006, the disclosures of which are incorporated herein by reference.

### FIELD OF THE INVENTION

The present invention relates to improved polymeric nanoparticles that are useful in delivery systems and the like to a patient

### SUMMARY OF THE INVENTION

There is an increasing demand for nanodevices, which are capable of carrying drugs to the targeted tissue or cells. Recently many drugs have been discovered, which show good efficiency in treatment of cancer or other diseases, however, their serious side effects cause difficult damages to the healthy tissues and organs. The targeted delivery of drugs and chemotherapies using nanodevices can protect the healthy part of the body and allow lower dosage for minimum inhibitory concentration (MIC). Nanodevices of the present invention with their sandwich like structure are able to protect the active ingredient carried and their surface is designed to avoid immune reactions. The nanodevices of the present invention are also designed to incorporate paramagnetic ions or metals for application as diagnostic contrast agents for Magnetic Resonance Imaging (MRI) and the like.

### BRIEF DESCRIPTION OF THE DRAWINGS

FIG. 1A is a representation of nanoparticles having a positively charged polyelectrolyte on the surface.

FIG. 1B shows a negatively charged polyelectrolyte on the surface of a nanoparticle.

FIG. 2 shows a schematic representation of an example of a nanodevice of the present invention.

FIG. 3 shows a schematic representation of a nanodevice conjugated with drug molecule.

FIG. 4 shows a schematic representation of a nanodevice conjugated with drug and targeting molecules.

FIG. 5 shows a schematic representation of a nanodevice conjugated with drug and targeting molecules, and containing MRI contrast agent. The drug molecule is e.g., MTX and the targeting molecule is e.g., folic acid. MRI contrast is due to presence of Gd³⁺ ions.

### DETAILED DESCRIPTION OF THE INVENTION

Macromolecules with ionizable functional groups such as carboxyl, amino, etc., in an aqueous medium form cations and anions, respectively. Under designed conditions of the present invention, polycations and polyanions form nanoparticles by ion-ion interactions. The formation of nanoparticles requires specific reaction parameters, otherwise flocculation and precipitation occurs. However, once the nanoparticles were formed at specific pH's and salt concentrations the nanosystem is stable.

### Sequence of Polyions

Ion-ion interaction can be performed between the functional groups of polyions, and the ratio of original polyions and the order of mixing can affect the sequence of the ion-ion interactions. The linear polyelectrolyte chains can collapse in a compact globule or can extend coil conformations depending on the pH of the reaction solution. The conformation of polymers is a factor in the sequence of polyelectrolyte.

Globules of nanoparticles can be formed, where the settlement of polyelectrolytes can be statistical. Core-shell or sandwich like morphology can be obtained by varying the ratio of original polyions, the pH and the order of mixing.

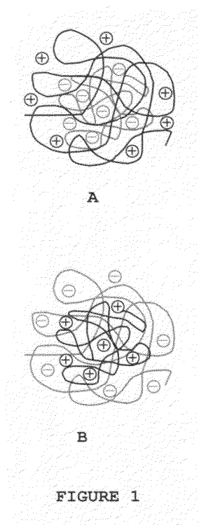

FIGS. 1A and B depicts a representation of nanoparticles formed by ion interaction of polyelectrolyte macromolecules. More specifically, FIG. 1A shows a positively charged polyelectrolyte (dark line) on the surface. FIG. 1B shows a negatively charged polyelectrolyte (light line) on the surface. The surface charge is determined by the sequence of mixing as described below.

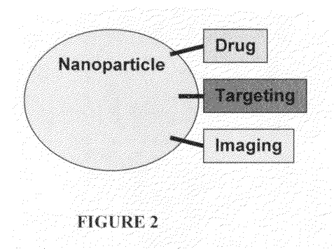

FIG. 2 shows a schematic representation of an example of a nanodevice of the present invention. The nanoparticle is conjugated with drug and targeting molecules, and with a function of imaging.

### Adjusting of pH

The size of nanoparticles depends on the pH of the solution. The hydrodynamic diameter of nanoparticles increases by increasing the pH.

### Surface Charge

Surface charge of nanoparticles can show the sequence of polyions. At lower pH, positively charged nanoparticles are typically formed independently of the ratio of polyions or order of mixing. By increasing the pH, negatively charged nanoparticles are formed, which show the charge of polyanions. The ratio of charged free functional groups determines the charge extent of nanoparticles, which depends on the pH and the ratio of functional groups.

### Salt Effect

The hydrodynamic diameter and the stability of nanoparticles were investigated in KCl solution. It was found that the hydrodynamic diameters decreased with increasing the salt concentration, but the stability of the aqueous solutions was independent of the salt concentration.

### Adjusting the Concentration of Polyions

The stability of the aqueous solution and the size of nanoparticles depend on the original concentration of polyions. The hydrodynamic diameter of nanoparticles increases with increasing the original concentration of polyions. The stability of the aqueous solution decreases with increasing the original concentrations, and precipitation can be observed in some cases of mixing at high concentration of original polyions.

### EXAMPLES

### Example 1

### Nanoparticles Formed from Poly Acrylic Acid (PAA) and Polyammonium salt (PAMM)

PAA with Mw=200 kDa and poly(2-methacryloxyethyltrimethylammonium bromide) were each separately dissolved in water at a concentration of 1 mg/ml. The pH value of solutions was adjusted to pH=3 by 0.10 mol/dm³ sodium hydroxide. To the solution of PAA under gentile stirring was added the solution of PAMM. After 1 hour the pH was increased to 7 resulting in a stable nanosystem with particle size of 50 to 350 nm measured by laser light scattering method. The size of nanoparticles may be varied and in a range of 10-1,000 nm by using polymers with different molecular weight. Also the particle size increases at higher pH due to the repulsion of negative charges.

### Example 2

### Nanoparticles Formed from Chitosan (CHIT) and Poly Gamma Glutamic Acid (PGA)

Chitosan is a linear polysaccharide of randomly distributed β-(1-4)-linked D-glucosamine (deacetylated unit) and N-acetyl-D-glucosamine (acetylated unit).

In the present example, CHIT with MW=320 kDa and PGA with Mw=1.3 MDa were each separately dissolved in water. The concentration of the solutions was varied in the range 0.1 mg/ml-2 mg/ml. The pH value of solutions was adjusted to pH=3 by 0.10 mol/dm³ hydrochloric acid. The ratio of polyelectrolyte and the order of mixing was modulated. After 1 hour mixing the pH was increased by 0.1 M sodium hydroxide solution resulting stable nanosystems. The hydrodynamic diameter of nanoparticles was in the range of 40-480 nm at pH=3, and at pH=7 was 470-1300 nm measured by laser light scattering method. There was some precipitation at higher pH caused by flocculation and coagulation. The size of nanoparticles may varied by using polymers with different molecular weight. By increasing the molecular weight of the polymers, the size of the nanoparticles similarly increases.

### Example 3

### Nanoparticles Formed from CHIT and Hyaluronic Acid (HYAL)

CHIT with Mv=320 kDa and HYAL with Mw=2.5 MDa were dissolved in water. The concentration of CHIT was varied in the range 0.1 mg/ml-1 mg/ml, and of HYAL 0.04-0.2 mg/ml. The pH value of solutions was adjusted to pH=3 by 0.10 mol/dm³ hydrochloric acid. The ratio of polyelectrolyte and the order of mixing was modulated. After 1 hour mixing the pH was increased by 0.1 M sodium hydroxide solution resulting stable nanosystems. The hydrodynamic diameter of nanoparticles was in the range of 130-350 nm at pH=3, and was higher than 600 nm at pH=7 measured by laser light scattering method. There are some precipitation at higher pH caused by flocculation and coagulation.

The size of nanoparticles may varied by using polymers with different molecular weight.

### Example 4

### Nanoparticles Formed from CHIT and Alginic Acid (ALGA)

CHIT with Mv=320 kDa and ALGA with Mv=30 kDa were dissolved in water. The concentration of CHIT was varied in the range 0.1 mg/ml-1 mg/ml, and of ALGA 0.04-0.2 mg/ml. The pH value of solutions was adjusted to pH=3 by 0.10 mol/dm³ hydrochloric acid. The ratio of polyelectrolyte and the order of mixing was modulated. After 1 hour mixing the pH was increased by 0.1 M sodium hydroxide solution resulting stable nanosystems at a pH=3. There are some precipitation at higher pH caused by flocculation and coagulation.

The size of nanoparticles may varied by using polymers with different molecular weight.

### Example 5

### Nanoparticles Formed from Modified CHIT and PGA

Chitosan was partially modified by betain. The modification was performed by using carbodiimide technique. CHIT was dissolved in hydrochloric acid media, betaine was dissolved in water and then adjusted the pH to 6.5 with 0.1 M sodium hydroxide solution. Water soluble carbodiimide was added to the betaine solution and the reaction was stirred for 30 min and subsequently mixed with chitosan solution.

The modified CHIT and PGA with Mw=1.3 MDa were dissolved in water. The concentration was varied in the range 0.1 mg/ml-2 mg/ml. The pH value of solutions was adjusted to pH=3 by 0.10 mol/dm³ hydrochloric acid. The ratio of polyelectrolyte and the order of mixing was modulated. After 1 hour mixing the pH was increased by 0.1 M sodium hydroxide solution resulting in stable nanosystems. There is some precipitation at higher pH caused by flocculation and coagulation. The size of nanoparticles may varied by using polymers with different molecular weight.

### Example 6

### Nanodevice for Delivery of DNA

CHIT with Mv=320 kDa was dissolved in water at pH=3. An aqueous solution of DNA with Mw=32 kDa and with a specific sequence was added. A stable nanosystem was formed. In the second step PGA with Mw-1.2 MDa was added to cover the residual surface. The sandwich like nanodevice containing the DNA molecules was stable at pH=7 and the NaCl concentration was 0.1 g/dm³.

### Example 7

### Nanodevice for Therapeutic Drug Delivery

CHIT with Mv was dissolved in an aqueous solution at pH=3. L-4-amino-N¹⁰-methylpteroyl-glutamic acid (L-amethopterin, MTX) as an anticancer drug was added. The pH was adjusted to a 4.5 value and PGA was added. Anticancer drug containing sandwich like nanodevice was formed, which was stable in the range of pH=6.5 to 7.5 and NaCl concentration was 0.9 g/dm³.

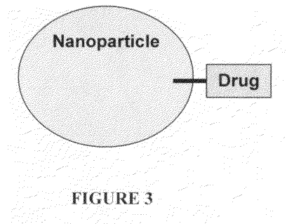

FIG. 3 shows a schematic representation of a nanodevice conjugated with drug molecule, where the drug molecule is e.g., MTX.

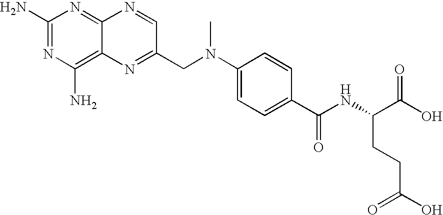

### Example 8

### Nanodevice for Targeted Therapeutic Drug Delivery

Nanodevice described in Example 7 was conjugated with folic acid as a targeting molecule for specific delivery to tumor cells.

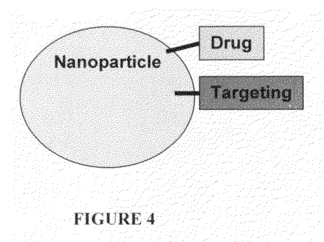

FIG. 4 shows a schematic representation of a nanodevice conjugated with drug and targeting molecules. where the drug molecule is e.g., MTX and the targeting molecule is e.g., folic acid.

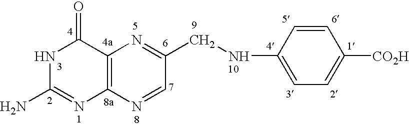

### Example 9

### Nanodevice for Targeted Therapeutic Drug Delivery and MRI Imaging

The nanodevice described in example 8 was modified with paramagnetic ion e.g., gadolinium ion. Gd³⁺ ion forms a complex PGA thus, under magnetic field the relaxation time of water molecules in the environment of nanodevices is different resulting in significant contrast.

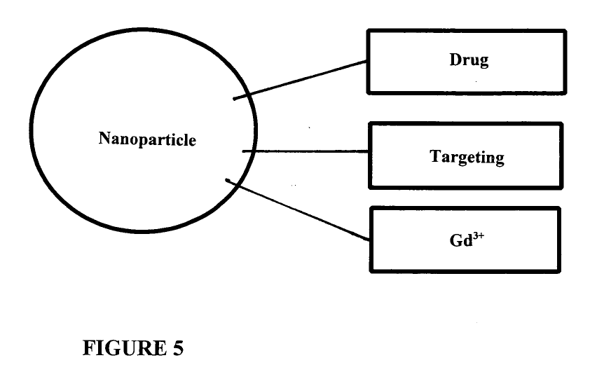

FIG. 5 shows a schematic representation of a nanodevice conjugated with drug and targeting molecules, and containing MRI contrast agent. The drug molecule is e.g., MTX and the targeting molecule is e.g., folic acid. MRI contrast is due to presence of Gd³⁺ ions.

### Example 10

In the present example, nanoparticles formed from chitosan (CHIT) and Poly y Glutamic Acid (PGA) are reacted with a peptide or a protein so that the peptide or a protein is bonded to the nanoparticle. Suitable peptides for this reaction include but are not limited to luteinizing hormone, releasing hormone (LHRH) and BCL-2 homology 3 (BH3).

The protein and/or peptide can be bonded to the nanoparticles by any suitable reaction process.

The protein or peptide is preferably bonded to the surface of the nanoparticle. Alternatively, the protein or peptide can be bonded to a nanoparticle that has been modified so that it is no longer globular or spherical and is more of a chain.

We claim:

1. A method of forming a carrier for delivery of an active ingredient to a patient undergoing treatment comprising
forming a first aqueous solution of a polyion wherein said polyion is either a positively charged ion or a negatively charged ion,
forming a second aqueous solution of said active ingredient and adding said second solution to said first aqueous solution to form a nanosystem
adding to said nanosystem an aqueous solution of a polyion having a charge opposite the charge of the polyion in the first aqueous solution,
said positively charged polyion and said negatively charged polyion forming a nanoparticle having said active ingredient between said positively and negatively charged ions.

2. The method according to claim 1 wherein said active ingredient is a drug.

3. The method according to claim 1 wherein said active ingredient is a nucleic acid.

4. The method according to claim 3 wherein said nucleic acid is RNA.

5. The method according to claim 3 wherein said nucleic acid is DNA.

6. The method according to claim 3 wherein said nucleic acid is siRNA.

7. The method according to claim 1 wherein the hydrodynamic diameter of the nanoparticles increases as the pH of the solution increases.

8. The method according to claim 1 wherein the active ingredient is folic acid.

9. The method according to claim 8 wherein said nanoparticle is modified with a paramagnetic ion to form a complex with the acid, said nanoparticle providing contrast under a magnetic field.

10. The method according to claim 9 wherein the paramagnetic ion is a gadolinium ion.

11. A method for forming a carrier for delivery of an active ingredient to a patient comprising:
in an aqueous solution blending a first polyion with an active ingredient to be delivered to said patient, said polyion being either a positively charged ion or a negatively charged ion,
adding to said aqueous solution a second polyion having a charge opposite the charge of the first polyion, said positively charged polyion and said negatively charged polyion forming a nanoparticle having said active ingredient between said positively charged and negatively charged ions whereby said polyions protect the active ingredient carried therein and wherein the surface covering of polyions prevents immune reactions with the active ingredient.

12. The method according to claim 11 wherein said active ingredient is a drug.

13. The method according to claim 11 wherein said active ingredient is a nucleic acid.

14. The method according to claim 13 wherein said nucleic acid is RNA.

15. The method according to claim 13 wherein said nucleic acid is DNA.

16. The method according to claim 13 wherein said nucleic acid is siRNA.

17. The method according to claim 11 wherein the hydrodynamic diameter of the nanoparticles increases as the pH of the solution increases.

18. The method according to claim 11 wherein the active ingredient is folic acid.

19. The method according to claim 18 wherein said nanoparticle is modified with a paramagnetic ion to form a complex with the polyacid, said nanoparticle providing contrast under a magnetic field.

20. The method according to claim 19 wherein the paramagnetic ion is a gadolinium ion.

21. A method of forming a nanoparticle comprising dissolving a first polyion in an aqueous solution, adding a drug to be delivered to a patient to said aqueous solution, adding a second polyion having a charge opposite the charge of the first polyion to form a nanoparticle reacting the nanoparticle so formed with a peptide or a protein to bond the peptide or protein to said nanoparticle.

22. The method according to claim 21 wherein said first polyion is chitosan.

23. The method according to claim 22 wherein said second polyion is poly gamma glutamic acid.

24. The method according to claim 24 wherein said peptide is a luteinizing hormone, releasing hormone (LHRH).

25. The method according to claim 24 wherein said protein is BCL-2 homology 3 (BH3).

26. The method according to claim 25 wherein said peptide or protein is bonded to the surface of the nanoparticle.
